(19) United States
(12) Patent Application Publication
(10) Pub. No.: US 2019/0187138 A1
(43) Pub. Date: Jun. 20, 2019

(54) SYSTEM FOR DETECTING CONCENTRATION OF AN ANALYTE BIOMOLECULE IN A SAMPLE AND METHOD THEREOF

(71) Applicant: Nanosniff Technologies Pvt. Ltd., Mumbai, Maharashtra (IN)

(72) Inventors: Nitin Kale, Mumbai (IN); Sumona Dhara, Thane (IN); Dadasaheb Sangave, Solapur (IN); Deepali Chandratre, Thane West (IN); Hrishikesh Desai, Thane West (IN); Nitin Mali, New Panvel (IN); Kapil Bardeja, Greater Noida (IN)

(21) Appl. No.: 15/742,026

(22) PCT Filed: Jun. 12, 2016
(86) PCT No.: PCT/IN2016/050233

§ 371 Date: Jan. 4, 2018

(30) Foreign Application Priority Data
Jul. 14, 2015 (IN) 2660/MUM/2015

(51) Int. Cl. G01N 33/543; G01N 27/12

(52) U.S. Cl. CPC G01N 33/54373; G01N 27/125; G01N 27/128

(57) ABSTRACT

Various embodiments of the methods and systems disclosed herein provide a detection system that detects concentration of an analyte biomolecule (e.g., a protein, a biomarker, etc.) in a sample (e.g., a blood sample) to diagnose a disease. The detection system includes one or more micro-cantilevers that are immobilized with one or more receptor biomolecules to detect a concentration of an analyte biomolecule in the sample. The detection system further compares the concentration of the analyte biomolecule with a threshold value for diagnosing a disease.

### BACKGROUND

### Technical Field

The embodiments herein generally relate to a system for detecting concentration of an analyte biomolecule in a sample, and, more particularly, to a system and method of detecting an analyte biomolecule from a sample using solid surfaces/micro-cantilevers that are coated with receptor biomolecules.

### Description of the Related Art

Non-communicable diseases such as ischemic heart diseases are the major problem affecting almost 17 million of the total world population (Yusuf et al., 2010). The global epidemiology of Acute myocardial infarction (AMI) was 33% in 2009 (WHO 2009) and it's been estimated to increase up to 46% by 2030 (Jamison D T, et al. 2006) with present Healthcare Facility. An early prognosis could save many lives. Also, survivors of myocardial infarction (MI) face a substantial high risk occurrence of further cardiovascular events thereby contributing to the increasing mortality rate. In addition, 50% of patients with AMI often do not exhibit any physical symptoms initially, which could be directly related to heart attack (Arrell et al., 2001). However, symptoms are evident with the increased amount of proteins released by the damaged myocardial cells in the blood stream after few hours or days of infarction. Hence, the non-symptomatic MI which has significant amount of detectable cardiac proteins in the blood stream can be referred as incipient or emerging cardiomyopathy (heart attack). The incipient heart attack is supposed to be an alarm or a wake-up call for the encroaching cardiac abnormalities (such as MI). Also, the characteristic symptom of MI is immense chest pain and dizziness. Quarter of the admitted heart attack patients does not have chest pain with normal EKG readings but mimic other symptomatic condition. This type of condition in a patient results in a “silent” myocardial infarctions. In addition, it is estimated that 64% of patients with previously diagnosed MI may have reoccurrence of cardiomyopathy which can be asymptomatic that is latent heart attack or symptomatic advanced heart attack. So, diagnosing a heart attack/MI is very difficult but the advent of immunoassaying techniques and piezoresistive properties of material aids in measuring the signal proteins in blood stream, help the physicians to identify the heart attack quickly and specifically.

Many of the existing techniques are quite slow in diagnosing a heart attack/MI. In addition, most of the aforementioned techniques do not lend themselves to measurement of changes in mass, which may provide a convenient way to measure a variety of different parameters. A mass sensor based on resonance frequency needs three components, an actuator (driver), a resonator, and a detector. A popular mass sensor is a silicon-based micro-cantilever due to its commercial availability and ease of integration with existing silicon based methodologies. In a silicon-based micro-cantilever mass sensor, the micro-cantilever acts as the resonator and is driven by an external lead zirconate titanate (PZT) actuator at the base of the micro-cantilever to generate vibrations in the resonator, which may be detected by an external optical detector. For bio-detection, receptors are immobilized on the micro-cantilever surface. Binding of antigens to the receptors immobilized on the cantilever surface increases the cantilever mass and causes a decrease in the resonance frequency. Detection of target molecules is achieved by monitoring the mechanical resonance frequency. In spite of the popularity of silicon-based micro-cantilevers, they rely on complex external optical components for detection. In addition, the PZT vibration driver adds to the weight and complexity of the sensor. Further, the external actuator can be located at the base of the micro-cantilever, which greatly limits its effectiveness in driving the cantilever's vibration. The optical means of detection also limits how small the micro-cantilever can be fabricated, and therefore limits the mass detection sensitivity.

In addition to mass detection, silicon-based micro-cantilevers have also been used as sensors for small molecules by detecting the stress generated on the cantilever by the adsorption of species onto receptors associated with the cantilever. Antibody or DNA receptors are coated on the surface of the micro-cantilevers to bind with target proteins or DNA molecules. The stress generated at the time of binding or unbinding of the target molecules to the receptors on the micro-cantilever surface induces a temporary deflection of the micro-cantilever that may be detected by external optical components or by an adsorption-stress-induced DC voltage due to a piezo-resistive layer embedded within the cantilever structure. Because the binding-induced stress decays with time, it can only be detected when the micro-cantilever is first introduced to the target molecules. The induced stress, and hence the induced DC voltage, dissipates quickly. Also, detecting the adsorption-induced stress in this manner offers no information about the amount of target antigen adsorbed on the cantilever.

Accordingly, there remains a need for a system to detect concentration of an analyte biomolecule in a sample (e.g., a blood sample) to diagnose a disease.

### SUMMARY

In view of the foregoing, an embodiment herein provides a system that detects an analyte biomolecule from a sample to diagnose a disease. The system includes a microfluidic chamber, a detection unit, an amplifier, an analog to digital converter, and a processing unit. The microfluidic chamber is configured to filter the sample to obtain a concentrated sample. The detection unit is configured to receive the concentrated sample from the microfluidic chamber. The detection unit includes one or more of detection micro-cantilevers, one or more of adjunct micro-cantilevers, and one or more of reference micro-cantilevers. The one or more of detection micro-cantilevers includes a first layer, and a second layer. The first layer of the one or more of detection micro-cantilevers is adapted to be immobilized with a one or more of first receptor biomolecules using an asymmetric site-specific and covalent immobilization process. The one or more of adjunct micro-cantilevers includes a first layer, and a second layer. The first layer of the one or more of adjunct micro-cantilevers is adapted to be immobilized with a one or more of second receptor biomolecules using the asymmetric site-specific, and covalent immobilization process. The second layer of (a) the one or more of detection micro-cantilevers, and (b) the one or more of adjunct micro-cantilevers are adapted to be coated with an amine blocker. The one or more of reference micro-cantilevers includes a first layer, and a second layer that are adapted to be coated with the amine blocker. The (a) one or more of detection micro-cantilevers, (b) one or more of adjunct micro-cantilevers, and (c) one or more of reference micro-cantilevers are adapted to be (i) supplied with a constant current, and (ii) exposed to the concentrated sample. Each of (a) one or more of detection micro-cantilevers, (b) one or more of adjunct micro-cantilevers, and (c) one or more of reference micro-cantilevers includes one or more piezo-resistive layers that are each embedded in between each of the first layer and the second layer of (a) the one or more of detection micro-cantilevers, (b) the one or more of adjunct micro-cantilevers, and (c) the one or more of reference micro-cantilevers. The detection unit is adapted to (i) measure a change in surface stress of at least one of (a) the one or more of detection micro-cantilevers, and (b) the one or more of adjunct micro-cantilevers due to binding of an analyte biomolecule with at least one of (c) the one or more of first receptor biomolecules, and (d) the one or more of second receptor biomolecules, and (ii) calculate a change in the resistance of at least one of (a) the one or more of detection micro-cantilevers, and (b) the one or more of adjunct micro-cantilevers. The detection unit calculates a change in voltage across (i) at least one of (a) the one or more of detection micro-cantilevers, and (b) the one or more of adjunct micro-cantilevers due to the change in the resistance of at least one of (a) the one or more of detection micro-cantilevers, and (b) the one or more of adjunct micro-cantilevers, and (ii) the one or more of reference micro-cantilevers. The amplifier is configured to (i) receive the voltages that correspond to at least one of (a) the one or more of detection micro-cantilevers, and (b) the one or more of adjunct micro-cantilevers, and (c) the one or more of reference micro-cantilevers, (ii) subtract the voltages that correspond to at least one of (a) the one or more of detection micro-cantilevers, and (b) the one or more of adjunct micro-cantilevers, and (c) the one or more of reference micro-cantilevers with voltages across the equivalent resistance of at least one of (a) the one or more of detection micro-cantilevers, and (b) the one or more of adjunct micro-cantilevers, and (c) the one or more of reference micro-cantilevers to obtain a one or more of differential voltages, and (iii) amplify the one or more of differential voltages to obtain a one or more of amplified differential voltages. The analog to digital converter is adapted to convert the one or more of amplified differential voltages into a digital signal. The processing unit is configured to process the digital signal to detect a concentration of the analyte biomolecule in the concentrated sample. The microfluidic chamber electrically isolates the concentrated sample from electrical contact pads of at least one of (a) the one or more of detection micro-cantilevers, and (b) the one or more of adjunct micro-cantilevers, and (c) the one or more of reference micro-cantilevers. In one embodiment, the processing unit is configured to compare the concentration of the analyte biomolecule that corresponds to at least one of (i) the one or more of first receptor biomolecules, and (ii) the one or more of second receptor biomolecules with a threshold value to diagnose a disease. In another embodiment, the microfluidic chamber includes (a) an inlet adapted to provide the sample to the detection unit, wherein the inlet includes a filter that is configured to filter the sample to obtain the concentrated sample, and (b) an outlet adapted to collect post-testing sample from the detection unit. In yet another embodiment, the processing unit determines the constant current that is supplied to (a) the one or more of detection micro-cantilevers, (b) the one or more of adjunct micro-cantilevers, and (c) the one or more of reference micro-cantilevers based on (a) a magnitude of the constant current required to detect and measure changes in surface activity, and (b) minimizing power dissipation. The constant current may range between 10 nanoamperes to 700 nanoamperes. In yet another embodiment, (i) the one or more of first receptor biomolecules, and (ii) the one or more of second receptor biomolecules is selected from at least one of (i) an antibody, (ii) a recombinant antibody, (iii) a protein, (iv) an antigen, (v) an enzyme, (vi) a nucleic acid, (vii) an oligonucleotide, (viii) an aptamers, (ix) a fragment of antibody, (x) a micro RNA, (xi) a modified mRNA, and (xii) a camelid. In yet another embodiment, the amine blocker is selected from at least one of (i) acid chloride, and (b) or anhydride.

In one aspect, a method of detecting a concentration of an analyte biomolecule from a sample is provided. The method includes the following steps: (i) immobilizing (a) a one or more of first receptor biomolecules on a first layer of a one or more of detection micro-cantilevers, and (b) a one or more of second receptor biomolecules on a first layer of a one or more of adjunct micro-cantilevers using an asymmetric site-specific, and covalent immobilization process; (ii) coating at least one of (i) acid chloride, and (ii) anhydride on (a) a second layer of the one or more of detection micro-cantilevers, (b) a second layer of the one or more of adjunct micro-cantilevers, and (c) a first layer and a second layer of a one or more of reference micro-cantilevers; (iii) supplying a constant current to (a) the one or more of detection micro-cantilevers, (b) the one or more of adjunct micro-cantilevers, and (c) the one or more of reference micro-cantilevers; (iv) exposing (a) the one or more of detection micro-cantilevers, (b) the one or more of adjunct micro-cantilevers, and (c) the one or more of reference micro-cantilevers to the sample; (v) measuring a change in surface stress of at least one of (a) the one or more of detection micro-cantilevers, and (b) the one or more of adjunct micro-cantilevers due to binding of an analyte biomolecule with at least one of (i) the one or more of first receptor biomolecules, and (ii) the one or more of second receptor biomolecule; (vi) calculating a change in the resistance of at least one of (a) the one or more of detection micro-cantilevers, and (b) the one or more of adjunct micro-cantilevers based on the change in surface stress of at least one of (a) the one or more of detection micro-cantilevers, and (b) the one or more of adjunct micro-cantilevers; (vii) calculating a change in voltage across (i) at least one of (a) the one or more of detection micro-cantilevers, and (b) the one or more of adjunct micro-cantilevers based on the change in resistance of at least one of (i) the one or more of detection micro-cantilevers, and (ii) the one or more of adjunct micro-cantilevers, and (ii) the one or more of reference micro-cantilevers; (viii) receiving the voltages that correspond to at least one of (a) the one or more of detection micro-cantilevers, and (b) the one or more of adjunct micro-cantilevers, and (c) the one or more of reference micro-cantilevers; (ix) subtracting the voltages that correspond to at least one of (a) the one or more of detection micro-cantilevers, and (b) the one or more of adjunct micro-cantilevers, and (c) the one or more of reference micro-cantilevers with voltages across the equivalent resistance of at least one of (a) the one or more of detection micro-cantilevers, and (b) the one or more of adjunct micro-cantilevers, and (c) the one or more of reference micro-cantilevers to obtain a one or more of differential voltages; (x) amplifying the one or more of differential voltages to obtain a one or more of amplified differential voltages; (xi) converting the one or more of amplified differential voltages into a digital signal; and (xii) processing the digital signal to detect a concentration of the analyte biomolecule in the concentrated sample. In one embodiment, the method further includes the step of comparing the concentration of the analyte biomolecule that corresponds to at least one of (i) the one or more of first receptor biomolecules, and (ii) the one or more of second receptor biomolecules with a threshold value to diagnose a disease.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A illustrates a block diagram of a detection system that detects a concentration of an analyte biomolecule in a sample to diagnose a disease according to an embodiment herein;

FIG. 1B illustrates an exploded view of a microfluidic chamber of FIG. 1 according to an embodiment herein;

FIG. 1C illustrates an exploded view of the (i) one or more detection micro-cantilevers, (ii) one or more adjunct micro-cantilevers, and (iii) the one or more reference micro-cantilevers of FIG. 1 according to an embodiment herein;

FIG. 1D illustrates an exploded view of layers of (i) one or more detection micro-cantilevers, or (ii) one or more adjunct micro-cantilevers of FIG. 1 according to an embodiment herein;

FIG. 2A illustrates a process of treating one or more antibodies with a 1-Ethyl-3-(3-dimethylaminopropyl)carbodiimide (EDC) according to an embodiment herein;

FIG. 2B illustrates a process of immobilizing site-specific EDC activated antibodies on a first layer of one or more micro-cantilevers of FIG. 1 according to an embodiment herein;

FIG. 2C illustrates a process of coating an amine blocker on a second layer of one or more amine functionalized micro-cantilevers according to an embodiment herein;

FIG. 3 is a flow diagram illustrating a process of immobilizing one or more receptor biomolecules on one or more solid surfaces according to an embodiment herein;

FIGS. 4A through 4C are flow diagrams illustrating a method of detecting a concentration of an analyte biomolecule in a sample using the detection system of FIG. 1 according to an embodiment herein;

FIG. 5A is a graphical representation that illustrates stability of an anti-FABP3 antibody immobilized micro-cantilever in a human serum sample according to an embodiment herein;

FIG. 5B is a graphical representation that illustrates response of an anti-Myoglobin antibody immobilized micro-cantilever, in the presence of human serum sample that includes hFABP3 protein, according to an embodiment herein;

FIG. 5C is a graphical representation that illustrates response of the antibody immobilized micro-cantilever, in presence of analyte biomolecule that is specific to immobilized antibody, according to an embodiment herein;

FIG. 5D is a graphical representation that illustrates response of (a) anti-Myoglobin antibody immobilized micro-cantilever, (b) anti-FABP3 antibody immobilized micro-cantilever, and (c) reference micro-cantilever, in presence of the human serum sample that includes Human FABP3 protein, according to an embodiment herein;

FIG. 6 illustrates an exploded view of a typical receiver according to an embodiment herein; and

FIG. 7 illustrates a schematic diagram of a computer architecture used in accordance with to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

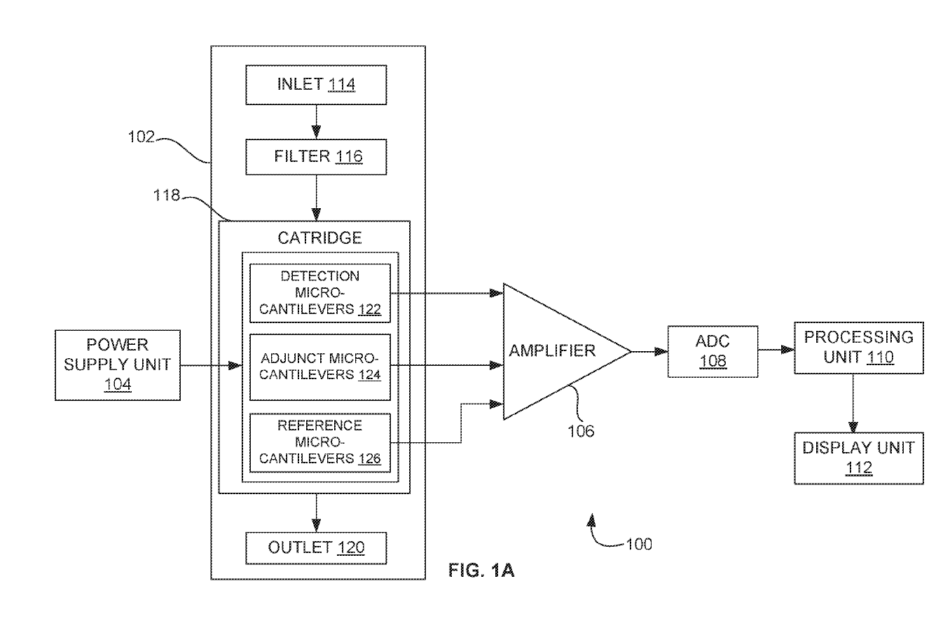

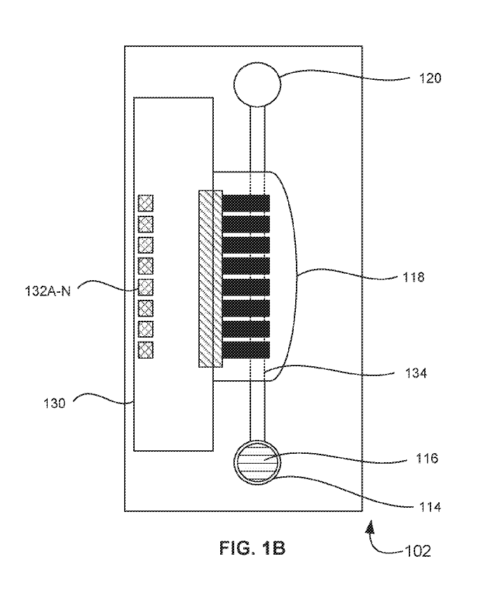

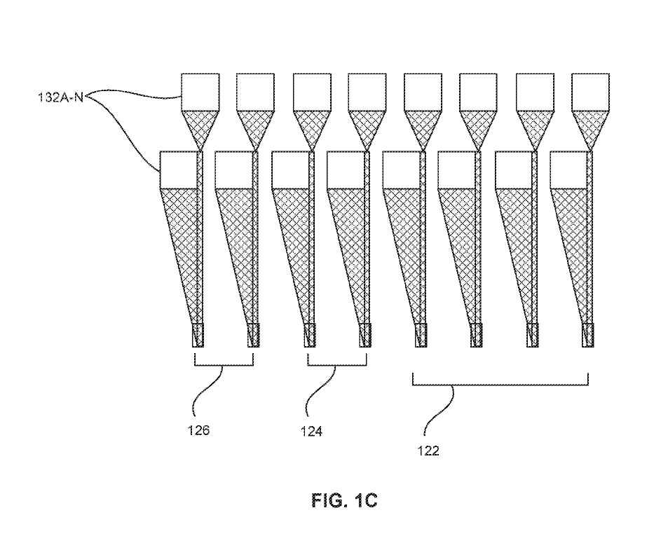

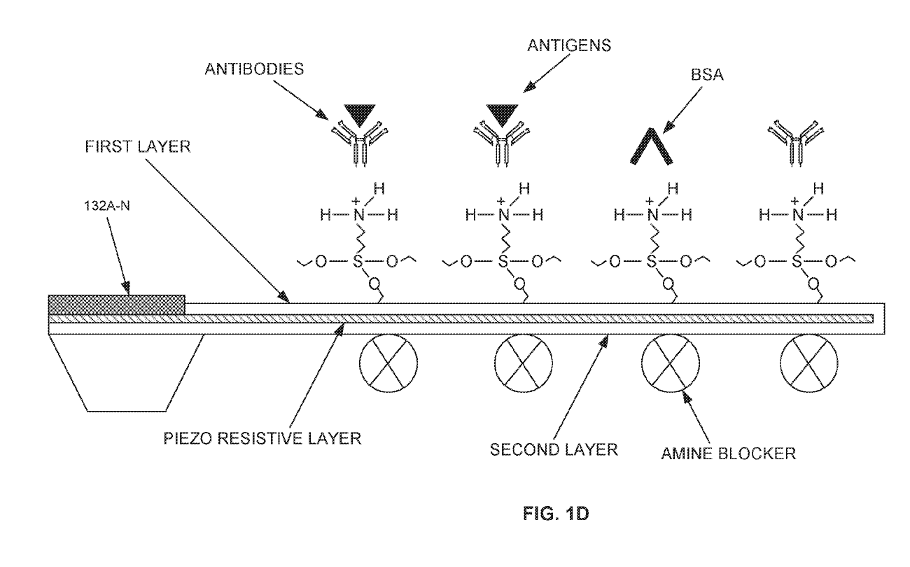

Various embodiments of the methods and systems disclosed herein provide a detection system that detects concentration of an analyte biomolecule (e.g., a protein, a biomarker, etc.) in a sample (e.g., a blood sample) to diagnose a disease. The detection system includes one or more micro-cantilevers that are immobilized with one or more receptor biomolecules to detect an analyte biomolecule in the sample for diagnosing a disease. Referring now to the drawings, and more particularly to FIGS. 1A through 7, where similar reference characters denote corresponding features consistently throughout the figures, there are shown preferred embodiments.

FIG. 1A illustrates a block diagram of a detection system 100 that detects a concentration of an analyte biomolecule in a sample to diagnose a disease according to an embodiment herein. The detection system 100 includes a microfluidic chamber 102, a power supply unit 104, an amplifier 106, an analog to digital converter (ADC) 108, a processing unit 110, and a display unit 112. The microfluidic chamber 102 includes an inlet 114, a filter 116, a detection unit 118, and an outlet 120. The detection unit 118 includes one or more detection micro-cantilevers 122, one or more adjunct micro-cantilevers 124, and one or more reference micro-cantilevers 126. The inlet 114 is adapted to provide a sample to the detection unit 118. The filter 116 is configured to filter the sample to obtain a concentrated sample. In one embodiment, the inlet 114 includes the filter 116 to filter the sample to obtain the concentrated sample. In another embodiment, the microfluidic chamber 102 provides a proper flow of the sample to the detection unit 118 (i.e. one or more detection micro-cantilevers 122, one or more adjunct micro-cantilevers 124, and one or more reference micro-cantilevers 126). The detection unit 118 receives the concentrated sample from the filter 116. The one or more detection micro-cantilevers 122 include a first layer (i.e. an immobilization layer), and a second layer (i.e. a structural layer). The first layer of the one or more detection micro-cantilevers 122 is adapted to be immobilized with one or more first receptor biomolecules (e.g., diagnostic receptor biomolecules, antibodies, enzymes, etc.) using an asymmetric, site-specific, covalent and uniform immobilization process. The one or more adjunct micro-cantilevers 124 include a first layer (i.e. an immobilization layer), and a second layer (i.e. a structural layer). The first layer of the one or more adjunct micro-cantilevers 124 is adapted to be immobilized with one or more second receptor biomolecules (e.g., prognostic and/or surrogate receptor biomolecules, antibodies, enzymes, etc.) using the asymmetric, site-specific, covalent and uniform immobilization process. The second layer of (a) the one or more detection micro-cantilevers 122, and (b) the one or more adjunct micro-cantilevers 124 are adapted to be coated with an amine blocker.

The one or more reference micro-cantilevers 126 include a first layer, and a second layer. The first layer and the second layer of the one or more reference micro-cantilevers 126 are adapted to be coated with the amine blocker. In one embodiment, the amine blocker may be an acid/acyl chloride, or anhydride. The acid chloride may be selected from at least one of (i) formyl chloride (CHClO), (ii) ethanoyl chloride (C₂H₃ClO), (iii) propanoyl chloride (C₃H₅ClO), (iv) butanoyl chloride (C₄H₇ClO), and (v) octanoyl chloride (C₈H₁₅ClO), etc. The solvent used for acid/acyl chloride may be chloroform or Dimethylformamide (DMF). The anhydride may be selected from at least one of (i) formic anhydride, (ii) ethanoic anhydride, (iii) propanoic anhydride, (iv) hexanoic anhydride, and (v) nonanoic anhydride, etc.

The one or more detection micro-cantilevers 122, the one or more adjunct micro-cantilevers 124, and the one or more reference micro-cantilevers 126 are adapted to be (i) supplied with a constant current, and (ii) exposed to the concentrated sample. In one embodiment, the one or more detection micro-cantilevers 122, the one or more adjunct micro-cantilevers 124, and the one or more reference micro-cantilevers 126 are pre-calibrated using calibrant solution. In another embodiment, the first layer, and the second layer of (i) the one or more detection micro-cantilevers 122, (ii) the one or more adjunct micro-cantilevers 124, and (iii) the one or more reference micro-cantilevers 126 may be an immobilization layer, and a structural layer respectively. In another embodiment, the first layer (i.e. the immobilization layer), and the second layer (i.e. the structural layer) of (i) the one or more detection micro-cantilevers 122, (ii) the one or more adjunct micro-cantilevers 124, and (iii) the one or more reference micro-cantilevers 126 are made up of dielectric material. In yet another embodiment, (i) the one or more detection micro-cantilevers 122, (ii) the one or more adjunct micro-cantilevers 124, and (iii) the one or more reference micro-cantilevers 126 may include a solid surface. The solid surface may be at least one of (a) natural polymers (i.e. cellulose, gelatin, etc), (b) synthetic polymers (e.g., polyvinyl chloride (PVC or vinyl)), (c) polystyrene, (d) polyethylene, (e) polypropylene, (f) polyacrylonitrile, (g) PVB, (h) silicone, (i) Cyclic olefin copolymer, (j) Polydimethylsiloxane (PDMS), (k) Poly(methyl methacrylate) (PMMA), (l) Polysulfone, (m) Polyimide, (n) acrylate, and (o) inorganic supports (e.g., silica, glass, silicon nitride, silicon oxide, gold, activated carbon).

The one or more detection micro-cantilevers 122, the one or more adjunct micro-cantilevers 124, and the one or more reference micro-cantilevers 126 include one or more piezo-resistive layers. The one or more piezo-resistive layers is embedded in between each of the immobilization layer (i.e. the first layer), and the structural layer (i.e. the second layer) of (a) the one or more detection micro-cantilevers 122, (b) the one or more adjunct micro-cantilevers 124, and (c) the one or more reference micro-cantilevers 126. The detection unit 118 is adapted to measure a change in surface stress of the one or more piezo-resistive layers of at least one of (a) the one or more detection micro-cantilevers 122, and (b) the one or more adjunct micro-cantilevers 124 due to binding of an analyte biomolecule (e.g., antigens, proteins, biomarkers, etc.) with at least one of (i) the one or more first receptor biomolecules (e.g., diagnostic receptor biomolecules), and (ii) the one or more second receptor biomolecules (e.g., prognostic and/or surrogate receptor biomolecules). In one embodiment, the one or more detection micro-cantilevers 122, and the one or more adjunct micro-cantilevers 124 bend either upward or downward when the surface stress of the one or more piezo-resistive layers change due to binding of an analyte biomolecule (e.g., antigens, proteins, etc.) with at least one of (i) the one or more first receptor biomolecules (e.g., diagnostic receptor biomolecules), and (ii) the one or more second receptor biomolecules (e.g., prognostic and/or surrogate receptor biomolecules).

The detection unit 118 is adapted to calculate a change in the resistance of at least one of (a) the one or more detection micro-cantilevers 122, and (b) the one or more adjunct micro-cantilevers 124 due to the change in surface stress of the one or more piezo-resistive layers of at least one of (a) the one or more detection micro-cantilevers 122, and (b) the one or more adjunct micro-cantilevers 124. In one embodiment, the detection unit 118 includes a digital potentiometer to calculate an equivalent resistance of at least one of (a) the one or more detection micro-cantilevers 122, and (b) the one or more adjunct micro-cantilevers 124, and (c) the one or more reference micro-cantilevers 126. In another embodiment, the digital potentiometer is adapted to be supplied with a constant current.

The detection unit 118 is adapted to calculate a change in voltage across (i) at least one of (a) the one or more detection micro-cantilevers 122, and (b) the one or more adjunct micro-cantilevers 124 based on the change in the resistance of at least one of (a) the one or more detection micro-cantilevers 122, and (b) the one or more adjunct micro-cantilevers 124, and (ii) the one or more reference micro-cantilevers 126. The detection unit 118 is further adapted to calculate voltages across the digital potentiometer based on the equivalent resistance of (a) the one or more detection micro-cantilevers 122, (b) the one or more adjunct micro-cantilevers 124, and (c) the one or more reference micro-cantilevers 126.

The amplifier 106 is configured to receive the voltages that correspond to (i) at least one of (a) the one or more detection micro-cantilevers 122, and (b) the one or more adjunct micro-cantilevers 124, and (c) the one or more reference micro-cantilevers 126, and (ii) the digital potentiometer. The amplifier 106 is configured to subtract the voltages that are received from at least one of (a) the one or more detection micro-cantilevers 122, (b) the one or more adjunct micro-cantilevers 124, and (c) the one or more reference micro-cantilevers 126 with the voltages received from the digital potentiometer to calculate one or more differential voltages. The amplifier 106 is configured to amplify the one or more differential voltage to obtain one or more amplified differential voltages. In one embodiment, the amplifier 106 may be a differential amplifier. The analog to digital converter (ADC) 108 is adapted to convert the one or more amplified differential voltages into a digital signal.

The processing unit 110 is configured to process the digital signal received from the ADC 108 to detect a concentration of the analyte biomolecule (e.g., antigens, proteins, biomarkers, etc.) in the concentrated sample. The processing unit 110 is configured to compare the concentration of the analyte biomolecule that corresponds to at least one of (i) the one or more first receptor biomolecules and (ii) the one or more second receptor biomolecules with a threshold value to diagnose a disease. In one embodiment, the processing unit 110 determines the constant current that is supplied to (a) the one or more detection micro-cantilevers 122, (b) the one or more adjunct micro-cantilevers 124, (c) the one or more reference micro-cantilevers 126, and (d) the digital potentiometer based on (a) a magnitude of the constant current required to detect and measure changes in surface activity, and (b) minimizing power dissipation. In another embodiment, the constant current ranges between 10 nanoamperes to 700 nanoamperes. The detection system 100 may include a display unit that is configured to display a status of one of (a) a disease is diagnosed, (b) no disease is diagnosed, or (c) the concentration of the analyte biomolecule.

In one embodiment, the one or more first receptor biomolecules (e.g., diagnostic receptor biomolecules), and the one or more second receptor biomolecules (e.g., prognostic and/or surrogate receptor biomolecules) are selected from at least one of (i) an antibody, (ii) a recombinant antibody, (iii) a protein, (iv) an antigen, (v) an enzyme, (vi) a nucleic acid, (vii) an oligonucleotide, (viii) an aptamers, (ix) a fragment of antibody, (x) a micro RNA, (xi) a modified mRNA, and (xii) a camelid. In yet another embodiment, the microfluidic chamber 102 electrically isolates the concentrated sample from electrical contact pads of at least one of (a) the one or more detection micro-cantilevers 122, and (b) the one or more adjunct micro-cantilevers 124, and (c) the one or more reference micro-cantilevers 126. The outlet 120 is adapted to collect post-testing sample from the detection unit 118. In one embodiment, the detection system 100 may be used by a qualified nurse/technician at a rural primary healthcare facility, or an ambulance. In another embodiment, the detection system 100 may be integrated with existing healthcare monitoring systems that are used in hospitals/health care centers.

An asymmetric immobilization process for immobilizing the first receptor biomolecules (e.g., diagnostic receptor biomolecules), and the second receptor biomolecules (e.g., prognostic and/or surrogate receptor biomolecules) on the one or more detection micro-cantilevers 122, and the one or more adjunct micro-cantilevers 124 respectively includes the following steps: (i) activating the one or more detection micro-cantilevers 122, and the one or more adjunct micro-cantilevers 124 using an oxidizing agent; (ii) treating the one or more detection micro-cantilevers 122, and the one or more adjunct micro-cantilevers 124 with aminosilane to obtain (a) one or more amine functionalized detection micro-cantilevers 122, and (b) one or more amine functionalized adjunct micro-cantilevers 124; (iii) treating (a) the first receptor biomolecules (e.g., diagnostic receptor biomolecules), and (b) the second receptor biomolecules (e.g., prognostic and/or surrogate receptor biomolecules) with a 1-Ethyl-3-(3-dimethylaminopropyl)carbodiimide (EDC) to obtain (a) a site-specific EDC activated first receptor biomolecules, and (b) a site-specific EDC activated second receptor biomolecules; and (iv) immobilizing (a) the site-specific EDC activated first receptor biomolecules, and (b) the site-specific EDC activated second receptor biomolecules on the first layer of (a) the one or more amine functionalized detection micro-cantilevers 122, and (b) the one or more amine functionalized adjunct micro-cantilevers 124. In one embodiment, the immobilizing step includes (i) treating (a) the site-specific EDC activated first receptor biomolecules, and (b) the site-specific EDC activated second receptor biomolecules with amine groups of (a) the first layer of the one or more amine functionalized detection micro-cantilevers 122, and (b) the first layer of the one or more amine functionalized adjunct micro-cantilevers 124 to form a covalent amide bond. In another embodiment, the 1-Ethyl-3-(3-dimethylaminopropyl)carbodiimide (EDC) is hydrolyzed from (a) the site-specific EDC activated first receptor biomolecules, and (b) the site-specific EDC activated second receptor biomolecules when (a) the site-specific EDC activated first receptor biomolecules, and (b) the site-specific EDC activated second receptor biomolecules bind with amine groups of (a) the one or more amine functionalized detection micro-cantilevers 122, and (b) the one or more amine functionalized adjunct micro-cantilevers 124.

The asymmetric immobilization process further includes the following steps: (i) coating a second layer of (a) the one or more amine functionalized detection micro-cantilevers 122, and (b) the one or more amine functionalized adjunct micro-cantilevers 124 with an amine blocker; (ii) washing (a) the one or more amine functionalized detection micro-cantilevers 122, and (b) the one or more amine functionalized adjunct micro-cantilevers 124 using organic solvents after treating (a) the one or more detection micro-cantilevers 122, and (b) the one or more adjunct micro-cantilevers 124 with the aminosilane; (iii) washing (a) the site-specific EDC activated first receptor biomolecules, and (b) the site-specific EDC activated second receptor biomolecules that are excess from (a) the one or more amine functionalized detection micro-cantilevers 122, and (b) the one or more amine functionalized adjunct micro-cantilevers 124 using at least one of (a) phosphate buffer saline, (b) de-ionised (DI) water after immobilizing (i) the site-specific EDC activated first receptor biomolecules, and (ii) the site-specific EDC activated second receptor biomolecules with (a) the one or more amine functionalized detection micro-cantilevers 122, and (b) the one or more amine functionalized adjunct micro-cantilevers 124; and (iv) treating (a) the one or more amine functionalized detection micro-cantilevers 122, and (b) the one or more amine functionalized adjunct micro-cantilevers 124 with bovine serum albumin to block unbound amine groups of (a) the one or more amine functionalized detection micro-cantilevers 122, and (b) the one or more amine functionalized adjunct micro-cantilevers 124. In one embodiment, the amine blocker may be an acid/acyl chloride, or anhydride.

In one embodiment, the asymmetric immobilization process further includes the following steps: (i) treating the one or more reference micro-cantilevers 126 with aminosilane to obtain one or more amine functionalized reference micro-cantilevers 126; (ii) coating a first layer and second layer of the one or more amine functionalized reference micro-cantilevers 126 with an amine blocker; and (iii) washing the one or more amine functionalized reference micro-cantilevers 126 using organic solvents after treating the one or more reference micro-cantilevers 126 with the aminosilane.

In an example embodiment, the detection system 100 detects an analyte biomolecule from a blood sample to diagnose an Acute Myocardial Infarction (AMI). The inlet 114 is adapted to provide a blood sample to the detection unit 118. The filter 116 is configured to filter red blood cell from the blood sample to obtain a concentrated sample. The detection unit 118 receives the concentrated sample from the filter 116. The detection unit 118 includes the one or more detection micro-cantilevers 122, the one or more adjunct micro-cantilevers 124, and the one or more reference micro-cantilevers 126. The one or more detection micro-cantilevers 122, and the one or more adjunct micro-cantilevers 124 include the immobilization layer (i.e. the first layer), and the structural layer (i.e. a second layer). The immobilization layer of the one or more detection micro-cantilevers 122 is adapted to be immobilized with first antibodies using an asymmetric, site-specific, covalent and uniform immobilization process. The first antibodies may be selected from at least one of (i) an anti-myoglobin antibody, (ii) an anti-FABP3 antibody, and (iii) an anti-troponin antibody (e.g., Troponin I, Troponin T, and Troponin C). The immobilization layer (i.e. the first layer) of the one or more adjunct micro-cantilevers 124 is adapted to be immobilized with second antibodies using the asymmetric, site-specific, covalent and uniform immobilization process. The second antibodies may be selected from at least one of (i) an anti-Human IgG antibody, (ii) an anti-IMA Antibody, (iii) an anti-Myeloperoxidase (MPO) Antibody, and (iv) an anti-Glycogen Phosphorylase Isoenzyme BB-(GPBB) Antibody. The structural layer (i.e. the second layer) of (a) the one or more detection micro-cantilevers 122, and (b) the one or more adjunct micro-cantilevers 124 are adapted to be coated with an amine blocker. The one or more reference micro-cantilevers 126 include the immobilization layer (i.e. the first layer), and the structural layer (i.e. the second layer). The immobilization layer and the structural layer of the one or more reference micro-cantilevers 126 are adapted to be coated with the amine blocker. In one embodiment, the amine blocker may be an acid/acyl chloride, or anhydride.

The one or more detection micro-cantilevers 122, the one or more adjunct micro-cantilevers 124, and the one or more reference micro-cantilevers 126 are adapted to be (i) supplied with a constant current, and (ii) exposed to the concentrated sample. The one or more detection micro-cantilevers 122, the one or more adjunct micro-cantilevers 124, and the one or more reference micro-cantilevers 126 include one or more piezo-resistive layers. The one or more piezo-resistive layers are embedded in between each of the immobilization layer (i.e. the first layer), and the structural layer (i.e. the second layer) of (a) the one or more detection micro-cantilevers 122, (b) the one or more adjunct micro-cantilevers 124, and (c) the one or more reference micro-cantilevers 126. The detection unit 118 is adapted to measure a change in surface stress of the one or more piezo-resistive layers of at least one of (a) the one or more detection micro-cantilevers 122, and (b) the one or more adjunct micro-cantilevers 124 due to binding of the analyte biomolecule (e.g., antigens, proteins, etc.) with at least one of (a) the first antibodies, and (b) the second antibodies. In one embodiment, the one or more detection micro-cantilevers 122, and the one or more adjunct micro-cantilevers 124 bend either upward or downward when the surface stress of the one or more piezo-resistive layers change due to binding of an analyte biomolecule (e.g., antigens, proteins, etc.) with at least one of (i) an anti-myoglobin antibody, (ii) an anti-FABP3 antibody, (iii) an anti-troponin antibody, (iv) an anti-Human IgG antibody, (v) an anti-IMA Antibody, (vi) an anti-Myeloperoxidase (MPO) Antibody, and (vii) an anti-Glycogen Phosphorylase Isoenzyme BB-(GPBB) Antibody.

The detection unit 118 is adapted to calculate a change in the resistance of at least one of (a) the one or more detection micro-cantilevers 122, and (b) the one or more adjunct micro-cantilevers 124 due to the change in surface stress of the one or more piezo-resistive layers of at least one of (a) the one or more detection micro-cantilevers 122, and (b) the one or more adjunct micro-cantilevers 124. The detection unit 118 includes a digital potentiometer to calculate an equivalent resistance of (a) the one or more detection micro-cantilevers 122, and (b) the one or more adjunct micro-cantilevers 124, and (c) the one or more reference micro-cantilevers 126. In one embodiment, the digital potentiometer is adapted to be supplied with a constant current.

The detection unit 118 is adapted to calculate a change in voltage across (i) at least one of (a) the one or more detection micro-cantilevers 122, and (b) the one or more adjunct micro-cantilevers 124 based on the change in the resistance of at least one of (a) the one or more detection micro-cantilevers 122, and (b) the one or more adjunct micro-cantilevers 124, and (ii) the one or more reference micro-cantilevers 126. The detection unit 118 is further adapted to calculate voltages across the digital potentiometer based on the equivalent resistance of (a) the one or more detection micro-cantilevers 122, and (b) the one or more adjunct micro-cantilevers 124, and (c) the one or more reference micro-cantilevers 126.

The amplifier 106 is configured to receive the voltages that correspond to (i) at least one of (a) the one or more detection micro-cantilevers 122, and (b) the one or more adjunct micro-cantilevers 124, and (c) the one or more reference micro-cantilevers 126, and (ii) the digital potentiometer. The amplifier 106 is configured to subtract the voltages that received from at least one of (a) the one or more detection micro-cantilevers 122, and (b) the one or more adjunct micro-cantilevers 124, and (c) the one or more reference micro-cantilevers 126, with the voltages received from the digital potentiometer to calculate one or more differential voltages. The amplifier 106 is configured to amplify the one or more differential voltage to obtain one or more amplified differential voltages. In one embodiment, the amplifier 106 may be a differential amplifier. The analog to digital converter (ADC) 108 is adapted to convert the one or more amplified differential voltages into a digital signal.

The processing unit 110 is configured to process the digital signal received from the ADC 108 to detect a concentration of the analyte biomolecule (e.g., antigens, proteins, etc.) in the concentrated sample. The processing unit 110 is configured to compare the concentration of the analyte biomolecule (e.g., antigens, proteins, etc.) that corresponds to at least one of (i) an anti-myoglobin antibody, (ii) an anti-FABP3 antibody, (iii) an anti-troponin antibody, (iv) an anti-Human IgG antibody, (v) an anti-IMA Antibody, (vi) an anti-Myeloperoxidase (MPO) Antibody, and (vii) an anti-Glycogen Phosphorylase Isoenzyme BB-(GPBB) Antibody with a threshold value to diagnose AMI.

In one embodiment, the processing unit 110 determines the constant current that is supplied to (a) the one or more detection micro-cantilevers 122, (b) the one or more adjunct micro-cantilevers 124, (c) the one or more reference micro-cantilevers 126, and the digital potentiometer based on (a) a magnitude of the constant current required to detect and measure changes in surface activity, and (b) minimizing power dissipation. In another embodiment, the constant current ranges between 10 nanoamperes to 700 nanoamperes. In yet another embodiment, the microfluidic chamber 102 is electrically isolates the concentrated sample from electrical contact pads of at least one of (a) the one or more detection micro-cantilevers 122, and (b) the one or more adjunct micro-cantilevers 124, and (c) the one or more reference micro-cantilevers 126. In one embodiment, the detection system 100 may be used by a qualified nurse/technician at a rural primary healthcare facility, or an ambulance. In another embodiment, the detection system 100 may be integrated with existing healthcare monitoring systems that are used in hospitals/health care centers.

With reference to FIG. 1A, FIG. 1B illustrates an exploded view of the microfluidic chamber 102 of FIG. 1 according to an embodiment herein. The microfluidic chamber 102 includes the detection unit 118, an isolation chamber 130, one or more electrical contact pads 132A-N, a fluid path 134. The detection unit 118 includes the one or more detection micro-cantilevers 122, the one or more adjunct micro-cantilevers 124, and the one or more reference micro-cantilevers 126. The isolation chamber 130 includes the one or more electrical contact pads 132A-N to power the one or more detection micro-cantilevers 122, the one or more adjunct micro-cantilevers 124, and the one or more reference micro-cantilevers 126. The inlet 114 is adapted to provide a sample to the detection unit 118. The filter 116 is configured to filter the sample to obtain a concentrated sample. The concentrated sample is given to the detection unit 118 through the fluid path 134. The detection unit 118 receives the concentrated sample from the filter 116. The one or more detection micro-cantilevers 122, the one or more adjunct micro-cantilevers 124, and the one or more reference micro-cantilevers 126 are adapted to be (i) supplied with a constant current through the electrical contact pads 132A-N, and (ii) exposed to the concentrated sample.

With reference to FIGS. 1A and 1B, FIG. 1C illustrates an exploded view of the (i) one or more detection micro-cantilevers 122, (ii) one or more adjunct micro-cantilevers 124, and (iii) the one or more reference micro-cantilevers 126 of FIG. 1 according to an embodiment herein. The one or more detection micro-cantilevers 122 include the first layer (i.e. the immobilization layer), and the second layer (i.e. the structural layer). The first layer of the one or more detection micro-cantilevers 122 is adapted to be immobilized with one or more first receptor biomolecules (e.g. antibodies) using an asymmetric, site-specific, covalent and uniform immobilization process. In one embodiment, the first layer of the one or more detection micro-cantilevers 122 is adapted to be immobilized with at least one of (i) an anti-myoglobin antibody, (ii) an anti-FABP3 antibody, and (iii) an anti-troponin antibody (e.g., Troponin I, Troponin T, and Troponin C). The one or more detection micro-cantilevers 122 are adapted to diagnose an Acute Myocardial Infarction. The one or more adjunct micro-cantilevers 124 include the first layer (i.e. the immobilization layer), and the second layer (i.e. the structural layer). The first layer of the one or more adjunct micro-cantilevers 124 is adapted to be immobilized with one or more second receptor biomolecules (e.g., antibodies) using the asymmetric, site-specific, covalent and uniform immobilization process. The second layer of (a) the one or more detection micro-cantilevers 122, and (b) the one or more adjunct micro-cantilevers 124 are adapted to be coated with an amine blocker. In one embodiment, the first layer of the one or more adjunct micro-cantilevers 124 is adapted to be immobilized with at least one of (i) an anti-Human IgG antibody, (ii) an anti-IMA Antibody, (iii) an anti-Myeloperoxidase (MPO) Antibody, and (iv) an anti-Glycogen Phosphorylase Isoenzyme BB-(GPBB) Antibody. The one or more adjunct micro-cantilevers 124 is adapted to diagnose an angina (i.e. chest pain). The one or more reference micro-cantilevers 126 include the first layer, and the second layer. The first layer and the second layer of the one or more reference micro-cantilevers 126 are adapted to be coated with the amine blocker. In one embodiment, the amine blocker may be an acid/acyl chloride, or anhydride. The one or more electrical contact pads 132A-N is adapted to power the one or more detection micro-cantilevers 122, the one or more adjunct micro-cantilevers 124, and the one or more reference micro-cantilevers 126.

With reference to FIGS. 1A through 1C, FIG. 1D illustrates an exploded view of layers of (i) one or more detection micro-cantilevers 122, or (ii) one or more adjunct micro-cantilevers 124 of FIG. 1 according to an embodiment herein. The one or more detection micro-cantilevers 122 include the first layer (i.e. the immobilization layer), and the second layer (i.e. the structural layer). The first layer of the one or more detection micro-cantilevers 122 is adapted to be immobilized with one or more first receptor biomolecules (e.g. antibodies) using an asymmetric, site-specific, covalent and uniform immobilization process. The one or more adjunct micro-cantilevers 124 include the first layer (i.e. the immobilization layer), and the second layer (i.e. the structural layer). The first layer of the one or more adjunct micro-cantilevers 124 is adapted to be immobilized with one or more second receptor biomolecules (e.g., antibodies) using the asymmetric, site-specific, covalent and uniform immobilization process. The second layer of (a) the one or more detection micro-cantilevers 122, and (b) the one or more adjunct micro-cantilevers 124 are adapted to be coated with an amine blocker. In another embodiment, the first layer (i.e. the immobilization layer), and the second layer (i.e. the structural layer) of (i) the one or more detection micro-cantilevers 122, and (ii) the one or more adjunct micro-cantilevers 124 are made up of dielectric material. The one or more electrical contact pads 132A-N to power the one or more detection micro-cantilevers 122, and the one or more adjunct micro-cantilevers 124. The one or more detection micro-cantilevers 122, and the one or more adjunct micro-cantilevers 124 include one or more piezo-resistive layers. The one or more piezo-resistive layers is embedded in between each of the immobilization layer (i.e. the first layer), and the structural layer (i.e. the second layer) of (a) the one or more detection micro-cantilevers 122, and (b) the one or more adjunct micro-cantilevers 124. The one or more detection micro-cantilevers 122, and the one or more adjunct micro-cantilevers 124 are adapted to be exposed to the concentrated sample. The one or more detection micro-cantilevers 122, and the one or more adjunct micro-cantilevers 124 bend either upward or downward due to binding of the analyte biomolecule (e.g., antigens, proteins, etc.) with at least one of (i) the one or more first receptor biomolecules (e.g., diagnostic receptor biomolecules), and (ii) the one or more second receptor biomolecules (e.g., prognostic and/or surrogate receptor biomolecules). The one or more detection micro-cantilevers 122, and the one or more adjunct micro-cantilevers 124 are treated with bovine serum albumin to block unbound amine groups of the one or more detection micro-cantilevers 122, and the one or more adjunct micro-cantilevers 124.

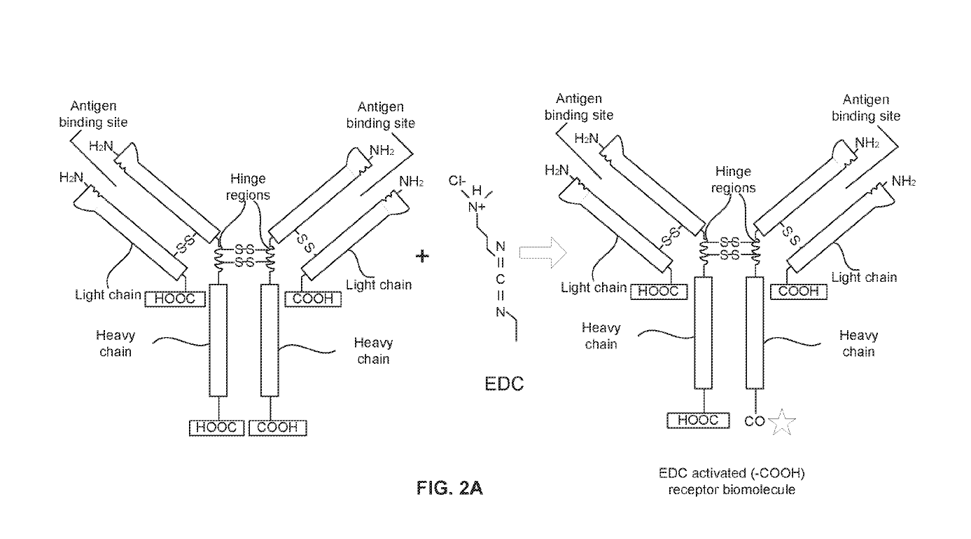

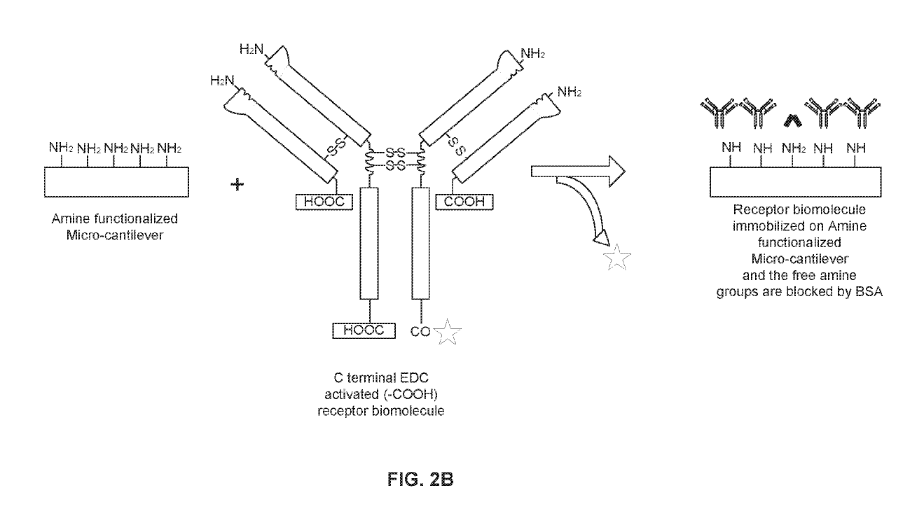

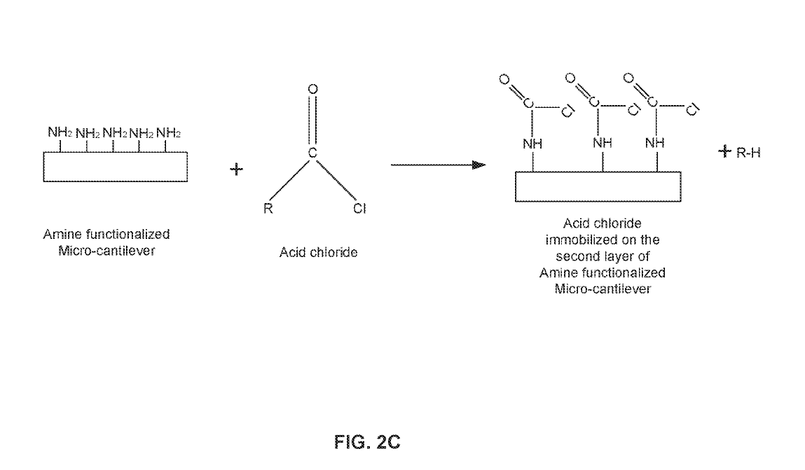

FIG. 2A illustrates a process of treating one or more antibodies with a 1-Ethyl-3-(3-dimethylaminopropyl)carbodiimide (EDC) according to an embodiment herein. The one or more antibodies (e.g., the first receptor biomolecules, or the second receptor biomolecules, etc.) are treated with a 1-Ethyl-3-(3-dimethylaminopropyl)carbodiimide (EDC) to obtain one or more site-specific EDC activated antibodies (e.g., one or more site-specific EDC activated first receptor biomolecules, and one or more site-specific EDC activated second receptor biomolecules). In one embodiment, the EDC is reacted with a C terminal of the one or more antibodies. In another embodiment, the one or more antibodies are selected from at least one of (i) a camelid, (ii) a recombinant antibody, (iii) a protein, (iv) an antigen, (v) an enzyme, (vi) a nucleic acid, (vii) an oligonucleotide, (viii) an aptamers, (ix) a fragment of antibody, (x) a micro RNA, and (xi) a modified mRNA.

With reference to FIG. 2A, FIG. 2B illustrates a process of immobilizing a site-specific EDC activated antibodies on the first layer of one or more micro-cantilevers according to an embodiment herein. The one or more micro-cantilevers (i.e. the one or more detection micro-cantilevers 122, the one or more adjunct micro-cantilevers 124, and the one or more reference micro-cantilevers 126) are activated using an oxidizing agent. In one embodiment, the oxidizing agent is selected from at least one of (i) Oxygen (O₂), (ii) Ozone (O₃), (iii) Hydrogen peroxide (H₂O₂), (iv) Nitric acid (HNO₃), (v) Sulfuric acid (H₂SO₄), and (vi) Sulphochromic acid, etc. The one or more micro-cantilevers are treated with aminosilane to obtain the one or more amine functionalized micro-cantilevers (i.e. the one or more amine functionalized detection micro-cantilevers 122, the one or more amine functionalized adjunct micro-cantilevers 124, and the one or more amine functionalized reference micro-cantilevers 126). The one or more site-specific EDC activated antibodies are immobilized on the first layer of the one or more amine functionalized micro-cantilevers. In one embodiment, the one or more site-specific EDC activated antibodies are treated with amine groups of the first layer of the one or more amine functionalized micro-cantilevers to form a covalent amide bond. In one embodiment, the 1-Ethyl-3-(3-dimethylaminopropyl)carbodiimide (EDC) is hydrolyzed from the one or more site-specific EDC activated antibodies when the one or more site-specific EDC activated antibodies binds with amine groups of the one or more amine functionalized micro-cantilevers.

With reference to FIGS. 2A and 2B, FIG. 2C illustrates a process of coating the amine blocker on the second layer of one or more amine functionalized micro-cantilevers according to an embodiment herein. The second layer of the one or more amine functionalized micro-cantilevers (i.e. the one or more amine functionalized detection micro-cantilevers 122, the one or more amine functionalized adjunct micro-cantilevers 124, and the one or more amine functionalized reference micro-cantilevers 126) are coated with the amine blocker. In one embodiment, the amine blocker may be an acid/acyl chloride, or anhydride. The acid chloride may be selected from at least one of (i) formyl chloride (CHClO), (ii) ethanoyl chloride (C₂H₃ClO), (iii) propanoyl chloride (C₃H₅ClO), (iv) butanoyl chloride (C₄H₇ClO), and (v) octanoyl chloride (C₈H₁₅ClO), etc. The solvent used for acid/acyl chloride may be chloroform or Dimethylformamide (DMF). The anhydride may be selected from at least one of (i) formic anhydride, (ii) ethanoic anhydride, (iii) propanoic anhydride, (iv) hexanoic anhydride, and (v) nonanoic anhydride, etc.

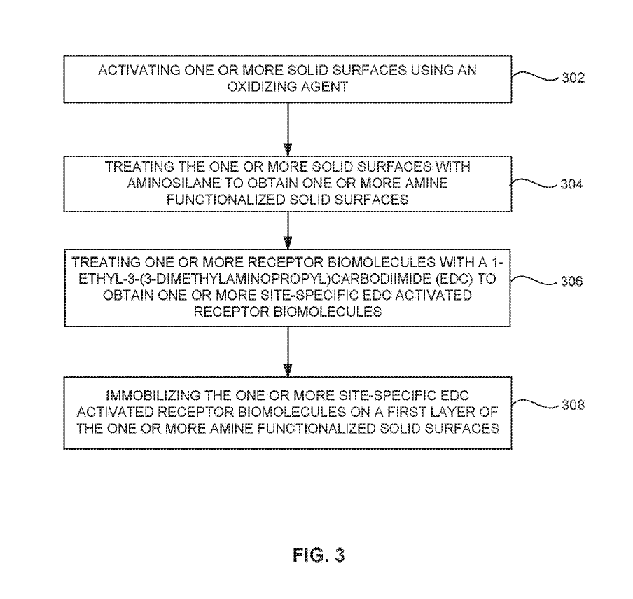

FIG. 3 is a flow diagram illustrating a process of immobilizing one or more receptor biomolecules on one or more solid surfaces according to an embodiment herein. At step 302, the one or more solid surfaces (i.e. the one or more detection micro-cantilevers 122, the one or more adjunct micro-cantilevers 124, and the one or more reference micro-cantilevers 126) are activated using an oxidizing agent. In one embodiment, the solid surfaces may be at least one of (a) natural polymers (i.e. cellulose, gelatin, etc), (b) synthetic polymers (e.g., polyvinyl chloride (PVC or vinyl)), (c) polystyrene, (d) polyethylene, (e) polypropylene, (f) polyacrylonitrile, (g) PVB, (h) silicone, (i) Cyclic olefin copolymer, (j) Polydimethylsiloxane (PDMS), (k) Poly(methyl methacrylate) (PMMA), (l) Polysulfone, (m) Polyimide, (n) acrylate, and (o) inorganic supports (e.g., silica, glass, silicon nitride, silicon oxide, gold, activated carbon). At step 304, the one or more solid surfaces are treated with aminosilane to obtain the one or more amine functionalized solid surfaces (i.e. the one or more amine functionalized detection micro-cantilevers 122, the one or more amine functionalized adjunct micro-cantilevers 124, and the one or more amine functionalized reference micro-cantilevers 126). At step 306, the one or more receptor biomolecules (e.g., the one or more first receptor biomolecules, and the one or more second receptor biomolecules) are treated with a 1-Ethyl-3-(3-dimethylaminopropyl)carbodiimide (EDC) to obtain one or more site-specific EDC activated receptor biomolecules (e.g., one or more site-specific EDC activated first receptor biomolecules, and one or more site-specific EDC activated second receptor biomolecules). At step 308, the one or more site-specific EDC activated receptor biomolecules are immobilized on the first layer of the one or more amine functionalized solid surfaces (e.g., the one or more amine functionalized detection micro-cantilevers 122, the one or more amine functionalized adjunct micro-cantilevers 124, and the one or more amine functionalized reference micro-cantilevers 126). The one or more site-specific EDC activated receptor biomolecules are treated with amine groups of the first layer of the one or more amine functionalized solid surfaces to form a covalent amide bond. In one embodiment, the second layer of the one or more amine functionalized solid surfaces are coated with the amine blocker. In another embodiment, the 1-Ethyl-3-(3-dimethylaminopropyl)carbodiimide (EDC) is hydrolyzed from the one or more site-specific EDC activated receptor biomolecules when the one or more site-specific EDC activated receptor biomolecules bind with amine groups of the one or more amine functionalized solid surfaces.

In another embodiment, the process further includes the following steps: (i) washing the one or more amine functionalized solid surfaces using organic solvents after treating the one or more solid surfaces with the aminosilane; (ii) washing the one or more site-specific EDC activated receptor biomolecules that are excess from the one or more amine functionalized solid surfaces using at least one of (a) phosphate buffer saline, (b) de-ionised water after immobilizing the one or more site-specific EDC activated receptor biomolecules on the one or more amine functionalized solid surfaces; and (iii) treating the first layer of the one or more amine functionalized solid surfaces with bovine serum albumin to block unbound amine groups of the one or more amine functionalized solid surfaces.

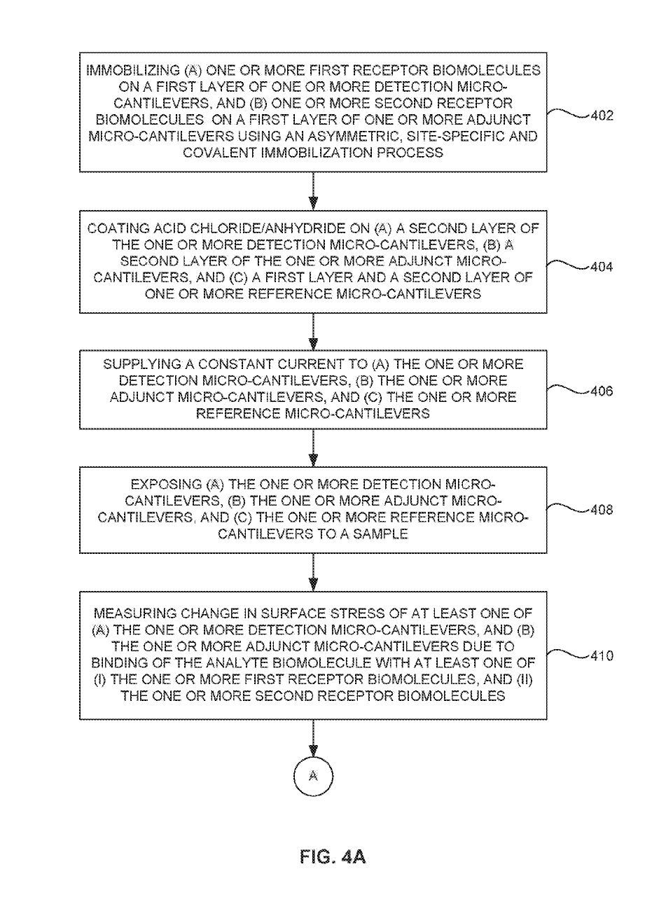

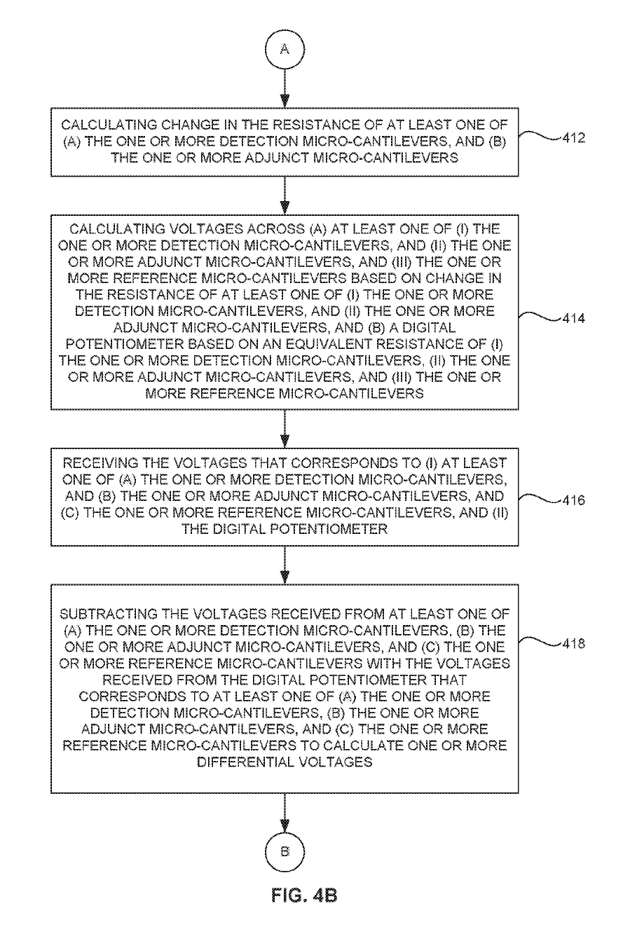

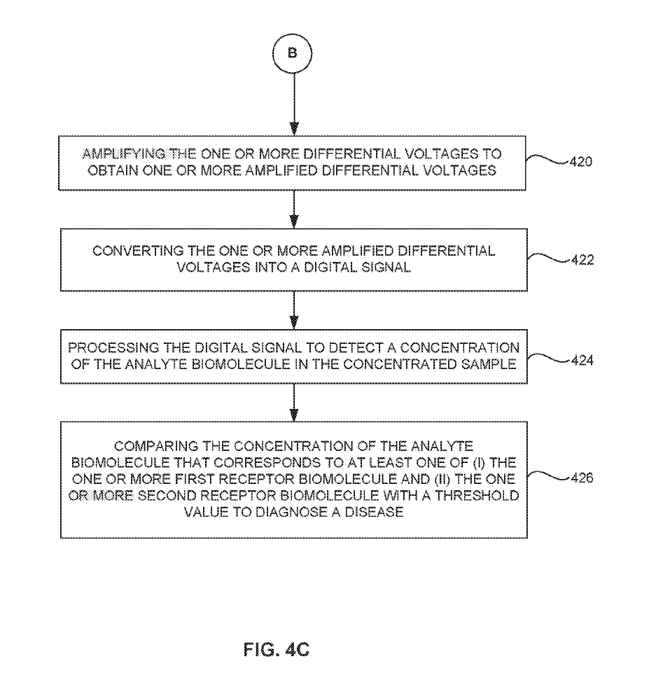

FIGS. 4A through 4C are flow diagrams illustrating a method of detecting a concentration of an analyte biomolecule in a sample using the detection system 100 of FIG. 1 according to an embodiment herein. At step 402, (a) the one or more first receptor biomolecules is immobilized on a first layer of one or more detection micro-cantilevers, and (b) the one or more second receptor biomolecules is immobilized on a first layer of one or more adjunct micro-cantilevers using an asymmetric, site-specific and covalent immobilization process. At step 404, (i) the second layer of (a) the one or more detection micro-cantilevers 122, and (b) the one or more adjunct micro-cantilevers 124, and (ii) the first layer and the second layer of the one or more reference micro-cantilevers 126 are adapted to be coated with an amine blocker. In one embodiment, the amine blocker may be an acid/acyl chloride, or anhydride. At step 406, the one or more detection micro-cantilevers 122, the one or more adjunct micro-cantilevers 124, and the one or more reference micro-cantilevers 126 are adapted to be supplied with a constant current. At step 408, the one or more detection micro-cantilevers 122, the one or more adjunct micro-cantilevers 124, and the one or more reference micro-cantilevers 126 are adapted to be exposed to the sample. At step 410, the detection unit 118 is adapted to measure a change in surface stress of at least one of (a) the one or more detection micro-cantilevers 122, and (b) the one or more adjunct micro-cantilevers 124 due to binding of the analyte biomolecule (e.g., antigens, proteins, etc.) with at least one of (i) the one or more first receptor biomolecules (e.g., antibodies), and (ii) the one or more second receptor biomolecules (e.g., antibodies). In one embodiment, the one or more piezo-resistive layers is embedded in between each of the immobilization layer (i.e. the first layer), and the structural layer (i.e. the second layer) of (a) the one or more detection micro-cantilevers 122, (b) the one or more adjunct micro-cantilevers 124, and (c) the one or more reference micro-cantilevers 126.

At step 412, the detection unit 118 is adapted to calculate a change in resistance of at least one of (a) the one or more detection micro-cantilevers 122, and (b) the one or more adjunct micro-cantilevers 124 due to the change in surface stress of at least one of (a) the one or more detection micro-cantilevers 122, and (b) the one or more adjunct micro-cantilevers 124. At step 414, the detection unit 118 is adapted to calculate a change in voltage across (i) at least one of (a) the one or more detection micro-cantilevers 122, and (b) the one or more adjunct micro-cantilevers 124 based on the change in the resistance of at least one of (a) the one or more detection micro-cantilevers 122, and (b) the one or more adjunct micro-cantilevers 124, and (ii) the one or more reference micro-cantilevers 126. The detection unit 118 is further adapted to calculate voltages across the digital potentiometer based on the equivalent resistance of (a) the one or more detection micro-cantilevers 122, (b) the one or more adjunct micro-cantilevers 124, and (c) the one or more reference micro-cantilevers 126. In one embodiment, the detection unit 118 includes a digital potentiometer to calculate an equivalent resistance of (a) the one or more detection micro-cantilevers 122, and (b) the one or more adjunct micro-cantilevers 124, and (c) the one or more reference micro-cantilevers 126. At step 416, the amplifier 106 is configured to receive the voltages that correspond to (i) at least one of (a) the one or more detection micro-cantilevers 122, and (b) the one or more adjunct micro-cantilevers 124, and (c) the one or more reference micro-cantilevers 126, and (ii) the digital potentiometer. At step 418, the amplifier 106 is configured to subtract the voltages that are received from at least one of (a) the one or more detection micro-cantilevers 122, (b) the one or more adjunct micro-cantilevers 124, and (c) the one or more reference micro-cantilevers 126 with the voltages received from the digital potentiometer to calculate one or more differential voltages.

At step 420, the amplifier 106 is configured to amplify the one or more differential voltage to obtain one or more amplified differential voltages. At step 422, the analog to digital converter (ADC) 108 is adapted to convert the one or more amplified differential voltages into a digital signal. At step 424, the processing unit 110 is configured to process the digital signal received from the ADC 108 to detect a concentration of the analyte biomolecule (e.g., antigens, proteins, etc.) in the sample. At step 426, the processing unit 110 is configured to compare the concentration of the analyte biomolecule (e.g., antigens, proteins, etc.) that corresponds to at least one of (i) the one or more first receptor biomolecules and (ii) the one or more second receptor biomolecules with a threshold value to diagnose a disease.

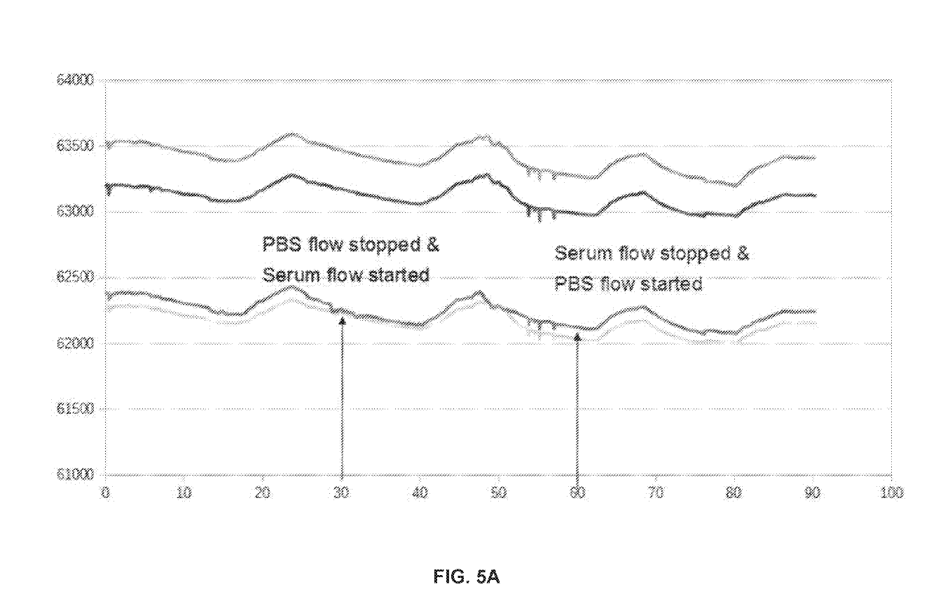

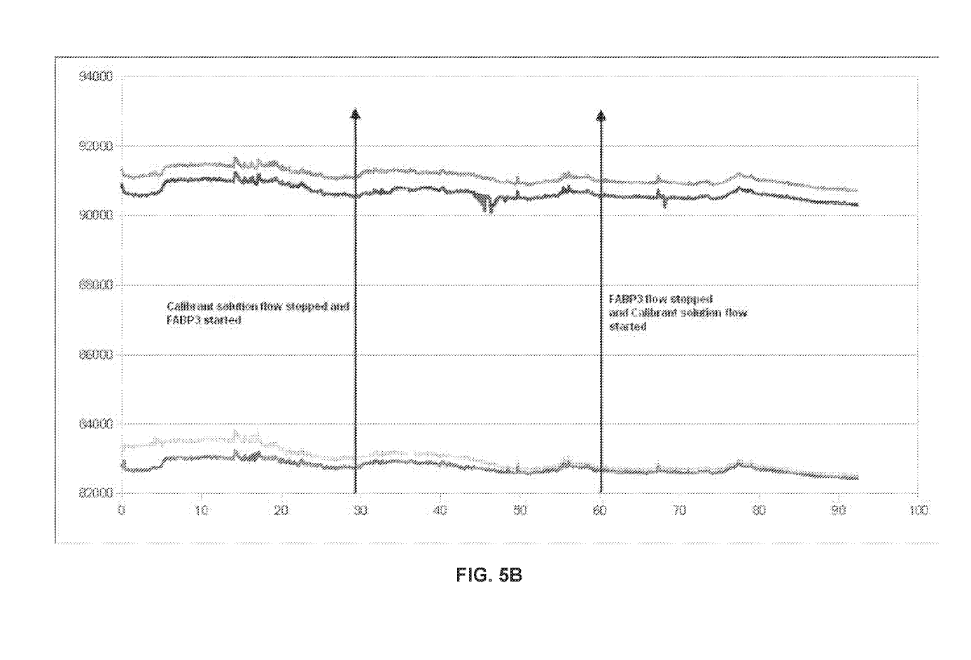

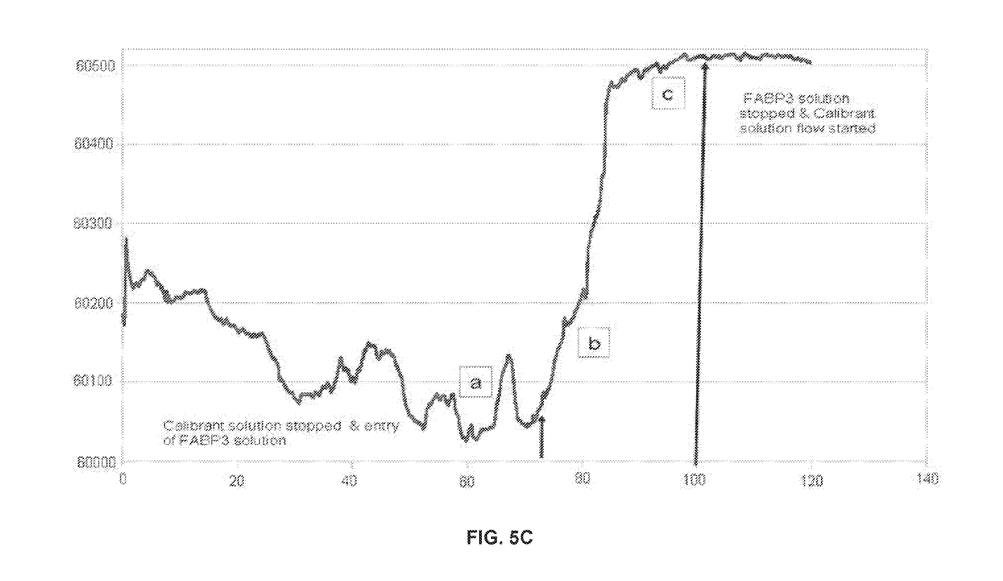

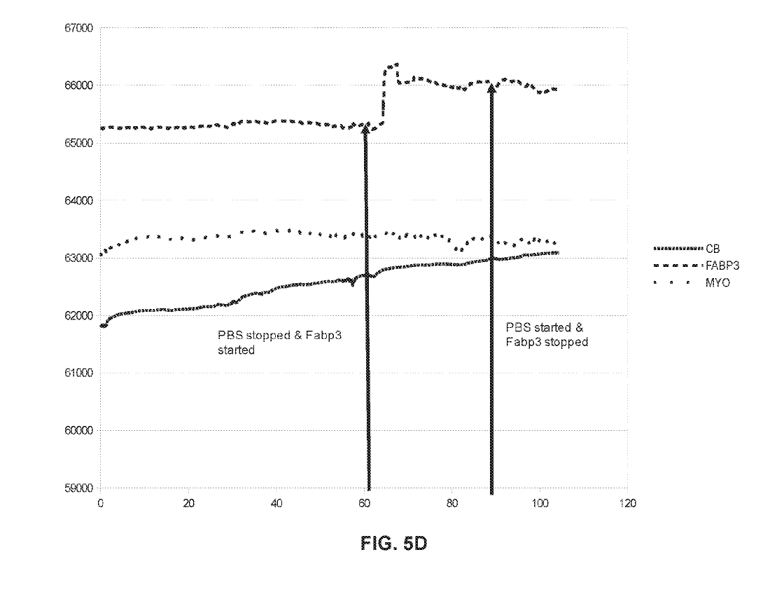

FIG. 5A is a graphical representation that illustrates stability of an anti-FABP3 antibody immobilized micro-cantilever in a human serum sample according to an embodiment herein. The graphical representation includes a X-axis and a Y-axis. The X-axis is plotted with time in minutes, and the Y-axis is plotted with resistance in ohms. Initially, the Anti-FABP3 antibody immobilized micro-cantilever is calibrated using Phosphate-buffered saline (PBS) to obtain PBS response. After calibration, the Anti-FABP3 antibody immobilized micro-cantilever is exposed to the human serum sample to obtain processed response. The processed response of the Anti-FABP3 antibody immobilized micro-cantilever is compared against the PBS response as shown in the graph. The graphical representation shows that Anti-FABP3 antibody immobilized micro-cantilever (e.g., detection micro-cantilevers 122), in presence of the human serum sample, shows no change in the resistance, despite the presence of bodily proteins, other than the FABP3 protein (i.e. antigen), in the human serum sample. Thus, the antibody-antigen complex is a specific and sensitive interaction.

FIG. 5B is a graphical representation that illustrates response of an anti-Myoglobin antibody immobilized micro-cantilever, in the presence of human serum sample that includes hFABP3 protein, according to an embodiment herein. The graphical representation includes a X-axis and a Y-axis. The X-axis is plotted with time in minutes, and the Y-axis is plotted with resistance in ohms. Initially, the Anti-Myoglobin antibody immobilized micro-cantilever is calibrated using Phosphate-buffered saline (PBS) to obtain PBS response. After calibration, the Anti-Myoglobin antibody immobilized micro-cantilever is exposed to the human serum sample that includes hFABP3 protein to obtain processed response. The processed response of the Anti-Myoglobin antibody immobilized micro-cantilever is compared against the PBS response as shown in the graph. The graphical representation shows that Anti-Myoglobin antibody immobilized micro-cantilever (e.g., detection micro-cantilevers 122), in presence of the human serum sample that includes hFABP3 protein, shows no change in the resistance, despite the presence of hFABP3 protein having molecular weight 14.9 kDa, which is almost equivalent to human myoglobin protein (16.7 kDa) in the human serum sample. Thus, the non-specific binding of antibody-antigen does not contribute to deflection of the micro-cantilever. When the anti-myoglobin antibody is immobilized on the micro-cantilever, and the human serum sample containing hFABP3 protein is injected, the micro-cantilever does not deflect. Moreover, the molecular weight/mass of proteins has no effect on the micro-cantilever deflection (i.e in producing the strain). In the above case, despite hFABP3 having molecular weight 14.9 kDa, which is almost equivalent to human myoglobin protein (16.7 kDa), the cantilever shows no change in the resistance.

FIG. 5C is a graphical representation that illustrates response of the antibody immobilized micro-cantilever, in presence of antigen/analyte biomolecule that is specific to immobilized antibody, according to an embodiment herein. The graphical representation includes a X-axis and a Y-axis. The X-axis is plotted with time in minutes, and the Y-axis is plotted with resistance in ohms. The antigen-antibody curve (i.e. sigmoid curve) shows three distinct stages (i.e. stage a, stage b, stage c). The stage a shows stable electrical response when the antigen (i.e. hFABP3 protein) is introduced to the anti-FABP3 antibody immobilized micro-cantilever (i.e. hFABP3 protein flow started). The stage b shows significant rise in electrical response due to deflection of the micro-cantilever (i.e. due to binding of anti-FABP3 antibody and hFABP3 protein). The stage c shows stability in electrical response due to saturation of the antibody-antigen reaction (i.e. anti-FABP3 antibody-hFABP3 protein reaction).

FIG. 5D is a graphical representation that illustrates response of (a) anti-Myoglobin antibody immobilized micro-cantilever, (b) anti-FABP3 antibody immobilized micro-cantilever, and (c) reference micro-cantilever, in presence of the human serum sample that includes Human FABP3 protein, according to an embodiment herein. The graphical representation includes a X-axis and a Y-axis. The X-axis is plotted with time in minutes, and the Y-axis is plotted with resistance in ohms. The graphical representation shows that Anti-FABP3 antibody immobilized micro-cantilever (e.g., detection micro-cantilevers 122), in presence of the human serum sample that includes human hFABP3 protein, shows change in the resistance/rise in electrical response due to deflection of Anti-FABP3 antibody immobilized micro-cantilever (i.e. due to binding of anti-FABP3 antibody and hFABP3 protein).

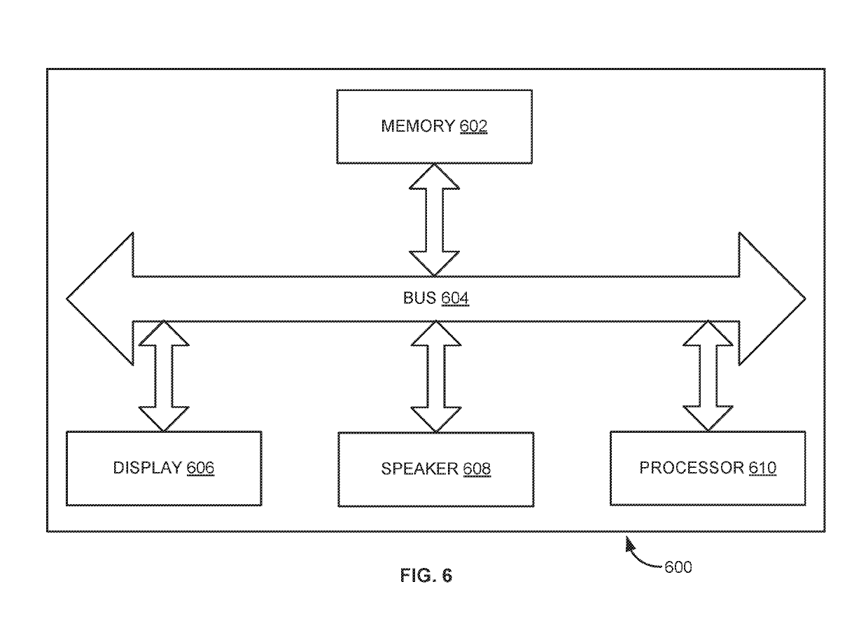

FIG. 6 illustrates an exploded view of a typical receiver 600 having an a memory 602 having a set of instructions, a bus 604, a display 606, a speaker 608, and a processor 610 capable of processing the set of instructions to perform any one or more of the methodologies herein, according to an embodiment herein. The processor 610 may also enable digital content to be consumed in the form of video for output via one or more displays 606 or audio for output via speaker and/or earphones 608. The processor 610 may also carry out the methods described herein and in accordance with the embodiments herein.

Digital content may also be stored in the memory 602 for future processing or consumption. The memory 602 may also store program specific information and/or service information (PSI/SI), including information about digital content (e.g., the detected information bits) available in the future or stored from the past. A user of the receiver 600 may view this stored information on display 606 and select an item of for viewing, listening, or other uses via input, which may take the form of keypad, scroll, or other input device(s) or combinations thereof. When digital content is selected, the processor 610 may pass information. The content and PSI/SI may be passed among functions within the receiver using the bus 604.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network). If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly.

The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

The embodiments herein can take the form of, an entirely hardware embodiment, an entirely software embodiment or an embodiment including both hardware and software elements. The embodiments that are implemented in software include but are not limited to, firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, remote controls, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

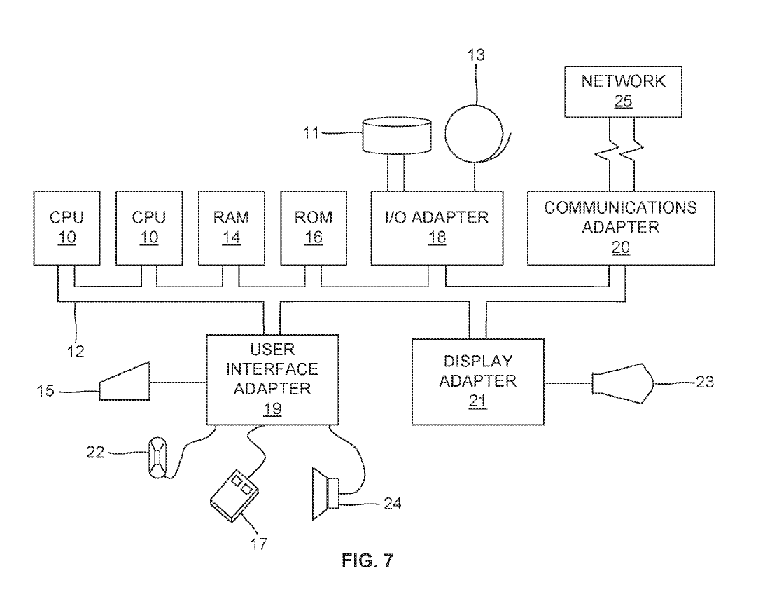

A representative hardware environment for practicing the embodiments herein is depicted in FIG. 7. This schematic drawing illustrates a hardware configuration of a computer architecture/computer system in accordance with the embodiments herein. The system comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system can read the inventive instructions on the program storage devices and follow these instructions to execute the methodology of the embodiments herein.

The system further includes a user interface adapter 19 that connects a keyboard 15, mouse 17, speaker 24, microphone 22, and/or other user interface devices such as a touch screen device (not shown) or a remote control to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

The detection system 100 includes one or more cantilevers (e.g., the one or more detection micro-cantilevers 122, and the one or more adjunct micro-cantilevers 124) are highly selective for one or more receptor biomolecule. The detection system 100 eliminates non-specific reaction between the receptor biomolecule and analyte biomolecule. The detection system 100 is highly sensitive, reliable, real-time, responsive, and easy to maintain. The detection system 100 consumes less power, and may be operated using a battery. The detection system 100 may detect both early and late markers (i.e. analyte biomolecules) to diagnose a disease (e.g., Myocardial infraction). The detection system 100 is an in-vitro, non-invasive, transducing device which is used to convert the strain generated due to receptor and analyte biomolecule reaction into a quantifiable signal.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

I/We claim:

1. A system that detects an analyte biomolecule from a sample to diagnose a disease, said system comprising:
a microfluidic chamber configured to filter the sample to obtain a concentrated sample;
a detection unit configured to receive the concentrated sample from the microfluidic chamber, wherein the detection unit comprises:
a plurality of detection micro-cantilevers, each comprising a first layer and a second layer, wherein the first layer of the plurality of detection micro-cantilevers is adapted to be immobilized with a plurality of first receptor biomolecules using an asymmetric, site-specific, and covalent immobilization process;
a plurality of adjunct micro-cantilevers that are positioned adjacent to the plurality of detection micro-cantilevers, wherein each of the plurality of adjunct micro-cantilevers comprises a first layer and a second layer, wherein the first layer of the plurality of adjunct micro-cantilevers is adapted to be immobilized with a plurality of second receptor biomolecules using the asymmetric, site-specific, and covalent immobilization process, wherein the second layer of (a) the plurality of detection micro-cantilevers, and (b) the plurality of adjunct micro-cantilevers is adapted to be coated with an amine blocker; and
a plurality of reference micro-cantilevers that are positioned adjacent to the plurality of adjunct micro-cantilevers, wherein each of the plurality of reference micro-cantilevers comprise a first layer and a second layer, wherein the first layer and the second layer of the plurality of reference micro-cantilevers are adapted to be coated with the amine blocker, wherein (a) the plurality of detection micro-cantilevers, (b) the plurality of adjunct micro-cantilevers, and (c) the plurality of reference micro-cantilevers are adapted to be (i) supplied with a constant current, and (ii) exposed to the concentrated sample, wherein each of (a) the plurality of detection micro-cantilevers, (b) the plurality of adjunct micro-cantilevers, and (c) the plurality of reference micro-cantilevers comprises:
a plurality of piezo-resistive layers that are each embedded in between each of the first layer and the second layer of (a) the plurality of detection micro-cantilevers, (b) the plurality of adjunct micro-cantilevers, and (c) the plurality of reference micro-cantilevers, wherein the detection unit is adapted to (i) measure a change in surface stress of at least one of (a) the plurality of detection micro-cantilevers, and (b) the plurality of adjunct micro-cantilevers due to binding of an analyte biomolecule with at least one of (c) the plurality of first receptor biomolecules, and (d) the plurality of second receptor biomolecules, and (ii) calculate a change in the resistance of at least one of (a) the plurality of detection micro-cantilevers, and (b) the plurality of adjunct micro-cantilevers, wherein the detection unit calculates a change in voltage across (i) at least one of (a) the plurality of detection micro-cantilevers, and (b) the plurality of adjunct micro-cantilevers due to the change in the resistance of at least one of (a) the plurality of detection micro-cantilevers, and (b) the plurality of adjunct micro-cantilevers, and (ii) the plurality of reference micro-cantilevers;
an amplifier configured to:
receive the voltages that correspond to at least one of (a) the plurality of detection micro-cantilevers, and (b) the plurality of adjunct micro-cantilevers, and (c) the plurality of reference micro-cantilevers;
subtract the voltages that correspond to at least one of (a) the plurality of detection micro-cantilevers, and (b) the plurality of adjunct micro-cantilevers, and (c) the plurality of reference micro-cantilevers with voltages across the equivalent resistance of at least one of (a) the plurality of detection micro-cantilevers, and (b) the plurality of adjunct micro-cantilevers, and (c) the plurality of reference micro-cantilevers to obtain a plurality of differential voltages; and
amplify the plurality of differential voltages to obtain a plurality of amplified differential voltages;
an analog to digital converter adapted to convert the plurality of amplified differential voltages into a digital signal; and
a processing unit configured to process the digital signal to detect a concentration of the analyte biomolecule in the concentrated sample,
wherein the microfluidic chamber electrically isolates the concentrated sample from electrical contact pads of at least one of (a) the plurality of detection micro-cantilevers, and (b) the plurality of adjunct micro-cantilevers, and (c) the plurality of reference micro-cantilevers.

2. The system as claimed in claim 1, wherein the processing unit is configured to compare the concentration of the analyte biomolecule that corresponds to at least one of (i) the plurality of first receptor biomolecules, and (ii) the plurality of second receptor biomolecules with a threshold value to diagnose a disease.

3. The system as claimed in claim 1, wherein the microfluidic chamber comprises:
an inlet adapted to provide the sample to the detection unit, wherein the inlet comprises a filter that is configured to filter the sample to obtain the concentrated sample; and
an outlet adapted to collect post-testing sample from the detection unit.

4. The system as claimed in claim 1, wherein the processing unit determines the constant current that is supplied to (a) the plurality of detection micro-cantilevers, (b) the plurality of adjunct micro-cantilevers, and (c) the plurality of reference micro-cantilevers based on (a) a magnitude of said constant current required to detect and measure changes in surface activity, and (b) minimizing power dissipation.

5. The system as claimed in claim 1, wherein (i) the plurality of first receptor biomolecules, and (ii) the plurality of second receptor biomolecules is selected from a group comprising:
(i) an antibody;
(ii) a recombinant antibody;
(iii) a protein;
(iv) an antigen;
(v) an enzyme;
(vi) a nucleic acid;
(vii) an oligonucleotide;
(viii) an aptamers;
(ix) a fragment of antibody;
(x) a micro RNA;
(xi) a modified mRNA; and
(xii) a camelid.

6. The system as claimed in claim 1, wherein the amine blocker is selected from at least one of (i) acid chloride, and (b) or anhydride.

7. A system that detects an analyte biomolecule from a sample to diagnose a disease, said system comprising:
a microfluidic chamber configured to filter the sample to obtain a concentrated sample;
a detection unit configured to receive the concentrated sample from the microfluidic chamber, wherein the detection unit comprises:
a plurality of detection micro-cantilevers, each comprising a first layer and a second layer, wherein the first layer of the plurality of detection micro-cantilevers is adapted to be immobilized with a plurality of first receptor biomolecules using an asymmetric, site-specific, and covalent immobilization process;
a plurality of adjunct micro-cantilevers that are positioned adjacent to the plurality of detection micro-cantilevers, wherein each of the plurality of adjunct micro-cantilevers comprises a first layer and a second layer, wherein the first layer of the plurality of adjunct micro-cantilevers is adapted to be immobilized with a plurality of second receptor biomolecules using the asymmetric, site-specific, and covalent immobilization process, wherein the second layer of (a) the plurality of detection micro-cantilevers, (b) the plurality of adjunct micro-cantilevers are adapted to be coated with at least one of (i) acid chloride, and (b) or anhydride; and
a plurality of reference micro-cantilevers that are positioned adjacent to the plurality of adjunct micro-cantilevers, wherein each of the plurality of reference micro-cantilevers comprises a first layer and a second layer, wherein the first layer and the second layer of the plurality of reference micro-cantilevers are adapted to be coated with at least one of (i) acid chloride, and (b) or anhydride, wherein (a) the plurality of detection micro-cantilevers, (b) the plurality of adjunct micro-cantilevers, and (c) the plurality of reference micro-cantilevers are adapted to be (i) supplied with a constant current, and (ii) exposed to the concentrated sample, wherein each of (a) the plurality of detection micro-cantilevers, (b) the plurality of adjunct micro-cantilevers, and (c) the plurality of reference micro-cantilevers comprises:
a plurality of piezo-resistive layers that are each embedded in between each of the first layer and the second layer of (a) the plurality of detection micro-cantilevers, (b) the plurality of adjunct micro-cantilevers, and (c) the plurality of reference micro-cantilevers, wherein the detection unit is adapted to (i) measure a change in surface stress of at least one of (a) the plurality of detection micro-cantilevers, and (b) the plurality of adjunct micro-cantilevers due to binding of an analyte biomolecule with at least one of (c) the plurality of first receptor biomolecules, and (d) the plurality of second receptor biomolecules, and (ii) calculate a change in the resistance of at least one of (a) the plurality of detection micro-cantilevers, and (b) the plurality of adjunct micro-cantilevers, wherein the detection unit calculates a change in voltage across (i) at least one of (a) the plurality of detection micro-cantilevers, and (b) the plurality of adjunct micro-cantilevers due to the change in the resistance of at least one of (a) the plurality of detection micro-cantilevers, and (b) the plurality of adjunct micro-cantilevers, and (ii) the plurality of reference micro-cantilevers;
an amplifier configured to:
receive the voltages that correspond to at least one of (a) the plurality of detection micro-cantilevers, and (b) the plurality of adjunct micro-cantilevers, and (c) the plurality of reference micro-cantilevers;
subtract the voltages that correspond to at least one of (a) the plurality of detection micro-cantilevers, and (b) the plurality of adjunct micro-cantilevers, and (c) the plurality of reference micro-cantilevers with voltages across the equivalent resistance of at least one of (a) the plurality of detection micro-cantilevers, and (b) the plurality of adjunct micro-cantilevers, and (c) the plurality of reference micro-cantilevers to obtain a plurality of differential voltages; and
amplify the plurality of differential voltages to obtain a plurality of amplified differential voltages;
an analog to digital converter adapted to convert the plurality of amplified differential voltages into a digital signal; and
a processing unit configured to:
process the digital signal to detect a concentration of the analyte biomolecule in the concentrated sample; and
compare the concentration of the analyte biomolecule that corresponds to at least one of (i) the plurality of first receptor biomolecules, and (ii) the plurality of second receptor biomolecules with a threshold value to diagnose a disease, wherein the processing unit determines the constant current that is supplied to (a) the plurality of detection micro-cantilevers, (b) the plurality of adjunct micro-cantilevers, and (c) the plurality of reference micro-cantilevers based on (a) a magnitude of said constant current required to detect and measure changes in surface activity, and (b) minimizing power dissipation,
wherein the microfluidic chamber electrically isolates the concentrated sample from electrical contact pads of at least one of (a) the plurality of detection micro-cantilevers, and (b) the plurality of adjunct micro-cantilevers, and (c) the plurality of reference micro-cantilevers.

8. The system as claimed in claim 7, wherein (i) the plurality of first receptor biomolecules, and (ii) the plurality of second receptor biomolecules is selected from a group comprising:
(i) an antibody;
(ii) a recombinant antibody;
(iii) a protein;
(iv) an antigen;
(v) an enzyme;
(vi) a nucleic acid;
(vii) an oligonucleotide;
(viii) an aptamers;
(ix) a fragment of antibody;
(x) a micro RNA;
(xi) a modified mRNA; and
(xii) a camelid.

9. A method of detecting a concentration of an analyte biomolecule from a sample, comprising:
immobilizing (a) a plurality of first receptor biomolecules on a first layer of a plurality of detection micro-cantilevers, and (b) a plurality of second receptor biomolecules on a first layer of a plurality of adjunct micro-cantilevers using an asymmetric, site-specific, and covalent immobilization process;
coating at least one of (i) acid chloride, and (ii) anhydride on (a) a second layer of the plurality of detection micro-cantilevers, (b) a second layer of the plurality of adjunct micro-cantilevers, and (c) a first layer and a second layer of a plurality of reference micro-cantilevers;
supplying a constant current to (a) the plurality of detection micro-cantilevers, (b) the plurality of adjunct micro-cantilevers, and (c) the plurality of reference micro-cantilevers;
exposing (a) the plurality of detection micro-cantilevers, (b) the plurality of adjunct micro-cantilevers, and (c) the plurality of reference micro-cantilevers to the sample;
measuring a change in surface stress of at least one of (a) the plurality of detection micro-cantilevers, and (b) the plurality of adjunct micro-cantilevers due to binding of an analyte biomolecule with at least one of (i) the plurality of first receptor biomolecules, and (ii) the plurality of second receptor biomolecules;
calculating a change in the resistance of at least one of (a) the plurality of detection micro-cantilevers, and (b) the plurality of adjunct micro-cantilevers based on the change in surface stress of at least one of (a) the plurality of detection micro-cantilevers, and (b) the plurality of adjunct micro-cantilevers;
calculating a change in voltage across (i) at least one of (a) the plurality of detection micro-cantilevers, and (b) the plurality of adjunct micro-cantilevers based on the change in resistance of at least one of (i) the plurality of detection micro-cantilevers, and (ii) the plurality of adjunct micro-cantilevers, and (ii) the plurality of reference micro-cantilevers;
receiving the voltages that correspond to at least one of (a) the plurality of detection micro-cantilevers, and (b) the plurality of adjunct micro-cantilevers, and (c) the plurality of reference micro-cantilevers;
subtracting the voltages that correspond to at least one of (a) the plurality of detection micro-cantilevers, and (b) the plurality of adjunct micro-cantilevers, and (c) the plurality of reference micro-cantilevers with voltages across the equivalent resistance of at least one of (a) the plurality of detection micro-cantilevers, and (b) the plurality of adjunct micro-cantilevers, and (c) the plurality of reference micro-cantilevers to obtain a plurality of differential voltages;
amplifying the plurality of differential voltages to obtain a plurality of amplified differential voltages;
converting the plurality of amplified differential voltages into a digital signal; and
processing the digital signal to detect a concentration of the analyte biomolecule in the concentrated sample.

10. The method as claimed in claim 9, wherein the method comprises comparing the concentration of the analyte biomolecule that corresponds to at least one of (i) the plurality of first receptor biomolecules, and (ii) the plurality of second receptor biomolecules with a threshold value to diagnose a disease.
